(19) United States
(12) Patent Application Publication
(10) Pub. No.: US 2005/0273032 A1
(43) Pub. Date: Dec. 8, 2005

(54) Filter and biosensor having the same

(76) Inventors: Atsunori Hiratsuka, Sagamihara (JP); Fumiaki Emoto, Hirakata (JP); Toshiaki Miyaji, Yokohama (JP)

Correspondence Address: WENDEROTH, LIND & PONACK, L.L.P., 2033 K STREET N. W., SUITE 800, WASHINGTON, DC, 20006-1021, US

(21) Appl. No.: 10/529,483

(22) PCT Filed: Apr. 26, 2004
(86) PCT No.: PCT/JP04/05994

§ 371 Date: Mar. 28, 2005

(30) Foreign Application Priority Data
Apr. 28, 2003 (JP) 2003-124049

(51) Int. Cl. A61B 19/00

(52) U.S. Cl. 604/6.15; 604/406

(57) ABSTRACT

The present invention relates to a filter for filtering a blood sample containing a blood cell component, which comprises a main body which defines a channel for causing the blood sample to flow, an opening for introducing the blood sample, the opening being located at one end of the channel, and an opening for discharging the blood sample filtered through the channel, the opening being located at the other end of the channel. A plurality of structures are disposed in the channel to prevent the blood cell component from passing through the channel. The structures are disposed at intervals such that a slit through which the blood cell component cannot pass is formed between each structure and an adjacent inner wall of the channel and between adjacent structures. The plurality of structures and the inner wall of the channel define at least one cavity functioning as a blood cell reservoir for accommodating the blood cell component in the channel.

### TECHNICAL FIELD

The present invention relates to a filter for separating blood components, which is used in clinical tests (particularly, point-of-care testing) in the fields of chemistry, biochemistry, medicine and the like, or at home, and a biosensor comprising the filter.

### BACKGROUND ART

Biochemical testing which measures a component in the blood is widely utilized for various kinds of diagnosis and observation and is an important testing method for clinical examination. Various biochemical testing devices have been developed to analyze a number of specimens or test items. In such testing, particular components in the blood (especially, the blood cell component) produce high background noise in measured values or interfere with the performance of measuring devices. Therefore, it is desirable to remove the blood cell component from samples. In most cases, testing devices are desired to be able to measure a small amount of sample.

Removal of the blood cell component is often performed using a general filter material. For example, there has been proposed a technology which relates to a sensor chip with built-in filter in which a general filter material (e.g., nonwoven fabric composed of hydrophilic fiber, such as glass fiber, cellulose or the like, pulp, filter paper, etc.) is provided in a pathway of an electrochemical sensor through which a blood sample is introduced (e.g., U.S. Patent Publication No. 2002/0148726).

However, in such a filter using a general filter material, a considerably amount of the blood plasma component is absorbed by the filter material, resulting in a considerably reduced amount of sample remaining after filtration. Therefore, a large amount of blood is required to obtain a sufficient amount of sample after filtration.

To address such a problem with the filter material, there have been developed devices in which a small amount of blood is used and the blood cell component is removed from the blood. For example, U.S. Pat. No. 6,319,719 discloses a blood cell component separation structure in which a sample is introduced into a pathway by capillary action and a number of obstructions having a crescent moon or bullet shape provided in the pathway are used to separate the blood cell component.

However, even in such a blood cell component separation structure which utilizes the capillary action and the obstructions in the pathway, there is still room for improving the required amount of blood. At the present time, sample volumes used in chip-type blood sugar sensors are about 0.3 to 4 μl. However, the blood cell component separation structure of U.S. Pat. No. 6,319,719 requires sample volumes of 20 to 50 μl for the filtration of blood. Therefore, a large amount of sample is still required as compared to when testing is performed using a blood sugar sensor without separation of blood cells.

A typical size of a blood sugar sensor is 6 mm in width and 10 mm in length. However, in the blood cell component separation structure of U.S. Pat. No. 6,319,719, the filtration of blood cells is achieved by the effect of delayed movement of blood cells, and therefore, the channel length needs to be great. Specifically, the capillary pathway (channel) needs to have a width of about 2 to 5 mm and a length of about 70 mm. The size of the device is large, and therefore, it is difficult to integrate the device with a chip-type blood sugar sensor.

In addition, the percentage of red blood cells in the blood is as large as about 50%. This characteristic of the blood that the component to be separated accounts for about 50% of the whole raises a particular problem that filters are likely to become clogged when used for separation of the blood cell component from the blood.

### DISCLOSURE OF THE INVENTION

The present invention is provided to solve the above-described conventional problems. An object of the present invention is to provide a blood component separation filter having a small size such that a considerably small amount of sample is required and the filter can be applied to a chip-type biosensor, and a biosensor integrated with such a filter. Another object of the present invention is to provide a filter which effectively resists clogging due to blood cell component, and a biosensor integrated with such a filter.

To achieve the object, the present invention provides a filter for filtering a blood sample containing a blood cell component. The filter comprises:
a channel for causing the blood sample to flow;
an opening for introducing the blood sample, the opening being located at one end of the channel; and
an opening for discharging the blood sample filtered through the channel, the opening being located at the other end of the channel,
wherein a plurality of structures are disposed in the channel to prevent the blood cell component from passing through the channel,
the structures are disposed at intervals such that a slit through which the blood cell component cannot pass is formed between each structure and an adjacent inner wall of the channel and between adjacent structures, and
the plurality of structures and the inner wall of the channel define at least one cavity functioning as a blood cell reservoir for accommodating the blood cell component in the channel.

In a preferable embodiment of the filter of the present invention, at least two cavities are defined in the channel.

In a preferable embodiment of the filter of the present invention, a depth of the cavity is greater than a width of a mouth of the cavity.

In a preferable embodiment of the filter of the present invention, a width of a mouth of the cavity is in a range of about 2 μm to about 10 μm.

In a preferable embodiment of the filter of the present invention, the cavity is in a shape of substantially a rectangular parallelepiped.

In a preferable embodiment of the filter of the present invention, a width of the slit is in a range of about 0.1 μm to about 2 μm.

In a preferable embodiment of the filter of the present invention, the channel is formed by a substrate, a spacer, and a cover attached to the substrate via the spacer.

In a preferable embodiment of the filter of the present invention, the structure is in a shape of a column.

In a preferable embodiment of the filter of the present invention, the structure is in a shape of a cylinder.

In a preferable embodiment of the filter of the present invention, the blood sample is introduced into the channel by capillary action.

In a preferable embodiment of the filter of the present invention, the structure and the inner wall of the channel are made of silicone resin, Teflon or epoxy resin, or surfaces of the structure and the inner wall of the channel are covered with any of silicone resin, Teflon and epoxy resin.

In another aspect, the present invention provides a biosensor having a filter region for filtering a blood sample containing a blood cell component. The biosensor comprises:
a substrate;
a measuring system supported by the substrate;
a reagent system containing a redox enzyme supported by the measuring system or the substrate in the vicinity of the measuring system;
a cover combined with the substrate to define, between the cover and the substrate, a filter region for removing the blood cell component from the blood sample, a reaction region for accommodating the measuring system and the reagent system, and a sample introduction pathway connected to the filter region for introducing the sample to the reaction region,
the filter region is defined by:
   a channel for causing the blood sample to flow;
   an opening for introducing the blood sample, the opening being located at one end of the channel;
   an opening for discharging the filtered blood sample, the opening being located at the other end of the channel and being connected to the sample introduction pathway; and
   a plurality of structures disposed in the channel to prevent the blood cell component from passing through the channel,
the structures are disposed at intervals such that a slit through which the blood cell component cannot pass is formed between each structure and an adjacent inner wall of the channel or between adjacent structures, and
the plurality of structures and the inner wall of the channel define at least one cavity functioning as a blood cell reservoir for accommodating the blood cell component in the channel.

In a preferable embodiment of the biosensor of the present invention, the measuring system includes an electrode system comprising at least a pair of electrodes.

In a preferable embodiment of the biosensor of the present invention, at least two cavities are defined in the channel.

In a preferable embodiment of the biosensor of the present invention, a depth of the cavity is greater than a width of a mouth of the cavity.

In a preferable embodiment of the biosensor of the present invention, a width of a mouth of the cavity is in a range of about 2 μn to about 10 μm.

In a preferable embodiment of the biosensor of the present invention, the cavity is in a shape of substantially a rectangular parallelepiped.

In a preferable embodiment of the biosensor of the present invention, a width of the slit is in a range of about 0.1 μm to about 2 μm.

In a preferable embodiment of the biosensor of the present invention, the channel is formed by a substrate, a spacer, and a cover attached to the substrate via the spacer.

In a preferable embodiment of the biosensor of the present invention, the structure is in a shape of a column.

In a preferable embodiment of the biosensor of the present invention, the structure is in a shape of a cylinder.

In a preferable embodiment of the biosensor of the present invention, the blood sample is introduced into the channel by capillary action.

In a preferable embodiment of the biosensor of the present invention, the structure and the inner wall of the channel are made of silicone resin, Teflon or epoxy resin, or surfaces of the structure and the inner wall of the channel are covered with any of silicone resin, Teflon and epoxy resin.

As used herein, the term “blood cell” or “blood cell component” has the same meaning as commonly understood by one of ordinary skill in the art, and refers to a red blood cell, a white blood cell and a blood platelet in the blood. However, only a red blood cell, only a white blood cell, or only a red blood cell and a white blood cell may be mainly considered as a “blood cell” or a “blood cell component”, depending on various situations, such as the purpose of examination or device design, since they have a particular influence on the accuracy of measurement results.

As used herein, the term “blood plasma” or “blood plasma component” has the same meaning as commonly understood by one of ordinary skill in the art, and refers to components (mainly serum and fibrinogen constituting the liquid component) in the blood excluding the blood cell component. Note that when blood platelet is not particularly considered as a “blood cell”, blood platelet is included in blood plasma or the blood plasma component (the same is true of other components in the “blood cell”).

The present invention provides a small-size filter capable of separating blood cells and blood plasma quickly using a trace amount of sample, and a biosensor comprising the filter.

According to one embodiment of the filter of the present invention, a blood sample can be introduced into a channel by capillary action. In this case, it is not necessary to apply pressure to the filter using a syringe or the like so as to cause the sample solution to flow, as is different from when a conventional filter material is used.

Also when a conventional filter material is used, the size of a pore formed by fiber or the like cannot be accurately determined (and is determined only as an average value). In the filter of the present invention, the size of a slit can be accurately determined. Therefore, by adjusting the slit width appropriately, it is possible to relatively easily select a molecule having a desired size from those having different sizes.

The filter of the present invention and a biosensor comprising the filter can be produced using a semiconductor processing technique, thereby making it possible to produce those simultaneously in large quantity and in uniform quality.

Typically, the present invention provides a very small biosensor with built-in filter having a width of about 5 mm, a length of about 9 mm, and a height of about 2.5 mm. By using the biosensor, a trace amount (about 30 nL) of blood can be separated into blood plasma and blood cells, and the glucose concentration or the like of the blood plasma can be measured. However, the technical scope of the present invention is not limited to such embodiments.

### BRIEF DESCRIPTION OF THE DRAWINGS

FIG. 1A shows a schematic top view of a blood component separation filter 1 according to an embodiment of the present invention which separates blood cells and blood plasma in the blood. FIG. 1B is a perspective view thereof.

FIG. 2A is a cross-sectional perspective view of the blood component separation filter 1 of the embodiment of the present invention, taken along a cross-section line shown in FIG. 1B. FIG. 2B is an enlarged top view of the inside of the channel in the filter 1 of FIG. 2A.

FIGS. 3A, 3B and 3C are schematic diagrams showing variations of column-shaped structures 19 and an arrangement thereof.

FIG. 4A is a schematic top view showing a structure of a biosensor 2 according to the present invention in which a filter for separating blood cells from collected blood is integrated with a biosensor for analyzing a test item, such as a blood sugar value or the like. FIG. 4B is a perspective view thereof.

FIG. 5A is a diagram showing a state of the filter 1 before introduction of a sample when the blood was filtered. FIG. 5B is a diagram showing a state of the filter 1 after introduction of the sample.

FIG. 6 is a diagram showing a change in response value of the biosensor 2 of the present invention with respect to glucose.

FIG. 7 is a diagram showing a change in response value of the biosensor 2 of the present invention with respect to cholesterol.

### BEST MODE FOR CARRYING OUT THE INVENTION

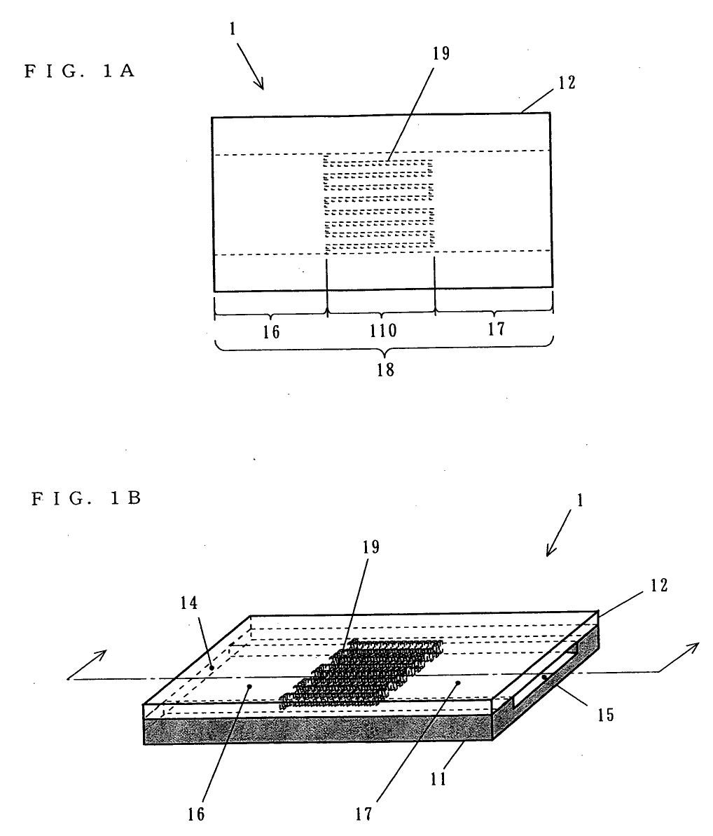

FIG. 1 shows a schematic top view (FIG. 1A) and a schematic perspective view (FIG. 1B) of a blood component separation filter 1 according to an embodiment of the present invention which separates blood cells and blood plasma.

Referring to FIGS. 1A and 1B, the blood component separation filter 1 of the present invention comprises a substrate 11 and a cover 12. A groove which serves as a channel is previously formed on the substrate 11. By attaching the cover 12 to the substrate 11, a channel 18 and a sample inlet 14 and a sample outlet 15 on both ends of the channel 18 are defined. The filter 1 further comprises a plurality of structures 19 for holding back blood cells on the channel 18.

In this embodiment, the minute structures 19 are arranged in the filter channel 18. Each structure 19 is preferably in the shape of a column, most preferably a cylinder. The structures 19 are spaced at appropriate intervals in the channel 19 so that blood cells do not pass therethrough. Typically, the column-shaped structures 19 can be produced by shaping the substrate 11 using a semiconductor processing technique, such as reactive ion etching or the like. By passing a blood sample through the thus-constructed filter 1, blood plasma and blood cells in the blood can be separated. In the above-described example, the substrate 11 is carved by etching to form the channel 18 and the structure 19. Alternatively, only the structure 19 may be produced by etching the substrate 11, and thereafter, a spacer may be disposed on both sides the structure 19 and the cover 12 may be attached to the substrate 11, thereby forming the channel 18.

As shown in FIG. 1, in the filter 1, a reservoir 16 for blood containing blood cells and a reservoir 17 for blood plasma are defined on respective sides (the sample inlet side and the sample outlet side) of a filter region 110 in which the column-shaped structure 19 is formed on the channel 18.

In a preferred embodiment of the blood component separation filter 1 of the present invention, a blood sample having a hematocrit value of 40 to 60 is used, and the sample is introduced by capillary action from the sample inlet 14 into the channel 18. Note that a hematocrit value indicates the percentage by volume of blood cells in the blood, typically the percentage by volume of red blood cells. Of the introduced blood components, blood cells are held back by a number of the column-shaped structures 19, and the remaining blood plasma is passed into the blood plasma reservoir 17 closer to the sample outlet 15. Thus, blood cells in the blood can be easily separated from the blood plasma component without using a syringe pump or the like. Note that the blood cells are not dissolved before and after the introduction of the blood.

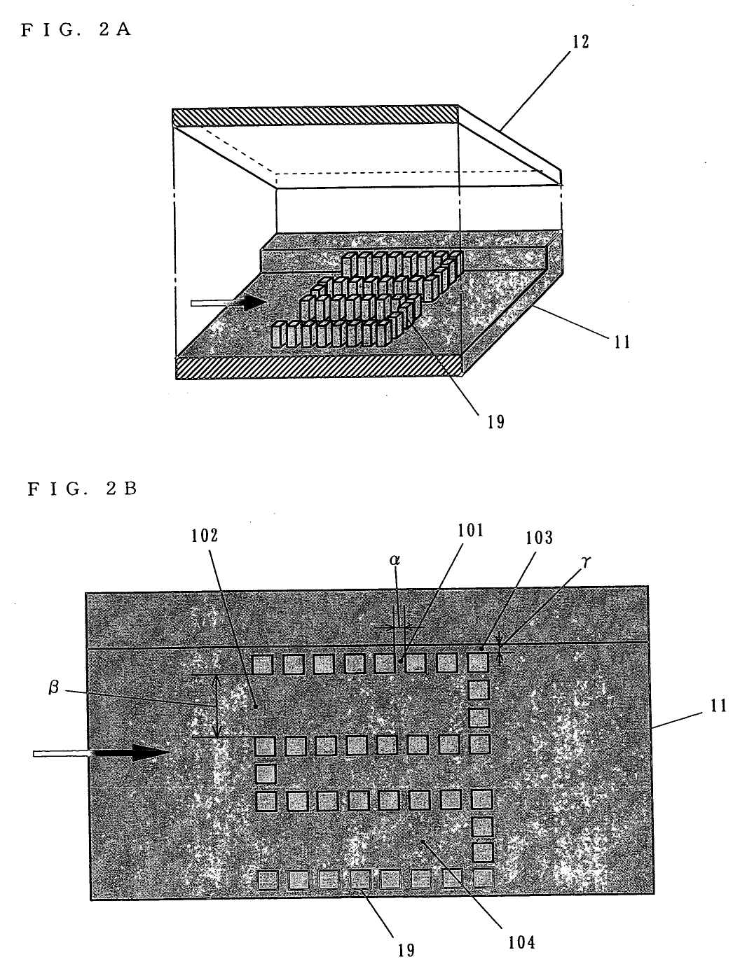

FIG. 2A is a cross-sectional perspective view of the blood component separation filter 1 of the embodiment of the present invention, taken along a cross-section line shown in FIG. 1B. Note that, in FIG. 2A, the cover 12 is separated from the substrate 11 for the sake of clarity of illustration, and the number and arrangement of the column-shaped structures 19 are simplified. An arrow in FIG. 2A indicates a direction in which a sample flows.

FIG. 2B is an enlarged top view of the inside of the channel in the filter 1 of FIG. 2A. An arrow indicates a direction in which a sample flows. As shown in FIG. 2B, a space between one column-shaped structure 19 and another adjacent column-shaped structure defines a slit 101, and a space between one column-shaped structure 19 and an inner wall of the channel 18 defines a slit 103.

The structures 19 cause only the blood plasma component to pass therethrough, but not blood cells. To achieve this, the structures 19 are arranged in the channel 18 in a manner such that the slit 101 and the slit 103 have an optimal width. The arrangement of the structures 19 has a folded portion so that a cavity 104 which functions as a blood cell reservoir for accommodating blood cells in association with the channel 18 is formed in the channel 18 as shown in FIG. 2B. Hereinafter, these structures will be described in more detail.

In the following description, a width of the slit 101 defined by a space between one column-shaped structure and another adjacent column-shaped structure is defined as α, and a width of the slit 103 defined by a space between the column-shaped structure 19 and the inner wall of the channel 18 is defined as γ. Also, a width of a mouth 102 of the cavity 104 formed by the column-shaped structures 19 and an inner wall of the channel 18 is defined as β.

The with α is determined so that the filter 1 of the present invention is given a function of holding back blood cells but passing the blood plasma component therethrough. A red blood cell has a flat disk shape and has an average thickness of about 2 μm and a diameter of about 8 μm. A white blood cell is a non-regular spherical molecule having a diameter of about 6 to about 25 μm. Therefore, the with α is preferably smaller than or equal to about 2 μm so that blood cells are held back and blood plasma flows easily. When α is smaller than about 0.1 μm, it is difficult even for the blood plasma component to pass through the slit 102 due to an action, such as liquid surface tension or the like. Therefore, α is preferably greater than or equal to about 0.1 μm. The result of an actual experiment on these characteristics is shown in Table 1.

TABLE 1

| | | | | | | | | | | |
|---|---|---|---|---|---|---|---|---|---|---|
| Space between | 8 | 4 | 2.2 | 2.0 | 1.8 | 1.6 | 1 | 0.3 | 0.1 | 0.05 |
| column-shaped | | | | | | | | | | |
| structures (α) | | | | | | | | | | |
| Were blood | x | x | x | ∘ | ∘ | ∘ | ∘ | ∘ | ∘ | Δ |
| cells held | | | | | | | | | | |
| back? | | | | | | | | | | |

Unit of values: μm
Legend of symbols: ◯: It was confirmed that blood cells were held back. x: It was not confirmed that blood cells were held back. Δ: Both blood cells and the liquid component were held back. Note: the width β of the mouth of the cavity 104 was 10 μm, a height of the mouth of the cavity 104 was 9 μm, and a cross-section of the column-shaped structure was a square of 2 μm×2 μm.

These optimal values for a hold true for the width γ between the column-shaped structure 19 and the inner wall of the channel 18. By setting the width α of the slit 101 between each column-shaped structure and the width γ of the slit 103 between the column-shaped structure and the inner wall of the channel as described above, it is possible to produce a blood cell separation filter which passes blood plasma, but not blood cells.

However, even if the slit widths are set as described above, “clogging” occurs in the slit 101 and the slit 103 since blood cells enter the slits as a blood sample flows. When clogging occurs, it is difficult even for blood plasma to pass through the filter, resulting in a reduction in efficiency of separation of blood plasma. To relax this phenomenon, in the filter 1 of the present invention, the arrangement of the column-shaped structures has a folded structure within the limited space in the channel as shown in FIGS. 1 and 2.

When the column-shaped structures 19 are arranged in a folded line within the channel 18, the number of slits through which the blood plasma component passes can be increased as compared to when the column-shaped structures 19 are arranged in a straight line within the channel 18. Further, the inner wall of the channel 18 and the array of the column-shaped structures 19 form the cavity 104 within the channel 18. The cavity 104 serves as a blood cell reservoir for accommodating blood cells, thereby making it possible to suppress blood cells from being accumulated in the reservoir 16 closer to the sample inlet. As a result, slit clogging can be suppressed, thereby increasing the efficiency of filtration.

Thus, a larger amount of the blood plasma component can pass through the slits quickly. Therefore, it is possible to separate blood components efficiently without an increase in the device size.

The width β of the mouth 102 of the cavity 104 created by the folded structure of the structures 19 and the inner wall of the channel 18 is preferably set to be greater than or equal to about 2 μm, taking into consideration that the thickness of a red blood cell is about 2 μm. If the width β is excessively small, a red blood cell cannot pass through the mouth 102. Note that the thickness of a red blood cell varies between sexes and among individuals, and therefore, the minimum value of the optimal β varies depending on the purpose.

On the other hand, the larger the width β, the lower the rate of recovery of the liquid component. This is because the percentage of the liquid component accumulated in the cavity is increased with an increase in the width β. Therefore, it is more efficient when the width β is not excessively great. The maximum value of the optimal β may be determined as appropriate by one of ordinary skill in the art, depending on the purpose. When blood cells are arranged in a line within the cavity 104, the most satisfactory blood cell separation efficiency is obtained (data not shown). Therefore, the width β is preferably smaller than or equal to about 10 μm which is substantially the same as the diameter of a red blood cell. The maximum value of the width β is most preferably smaller than or equal to about 8 μm.

Therefore, β is preferably between about 2 μm and about 10 μm, most preferably between about 2 μm and about 8 μm. However, the maximum value of the optimal β may be determined as appropriate by one of ordinary skill in the art, depending on the purpose.

The greater the depth of the cavity 104, the higher the filtration efficiency of blood plasma. However, the size of the cavity 104 is inevitably limited by the size of the main body of the filter 1 or the channel 18, and therefore, an optimal depth is determined as appropriate, depending on a desired filter size. Typically, for example, when the chip (filter main body) has a length of about 10 mm, a cavity having a depth of about 2 mm can be provided. The present invention is not limited to this.

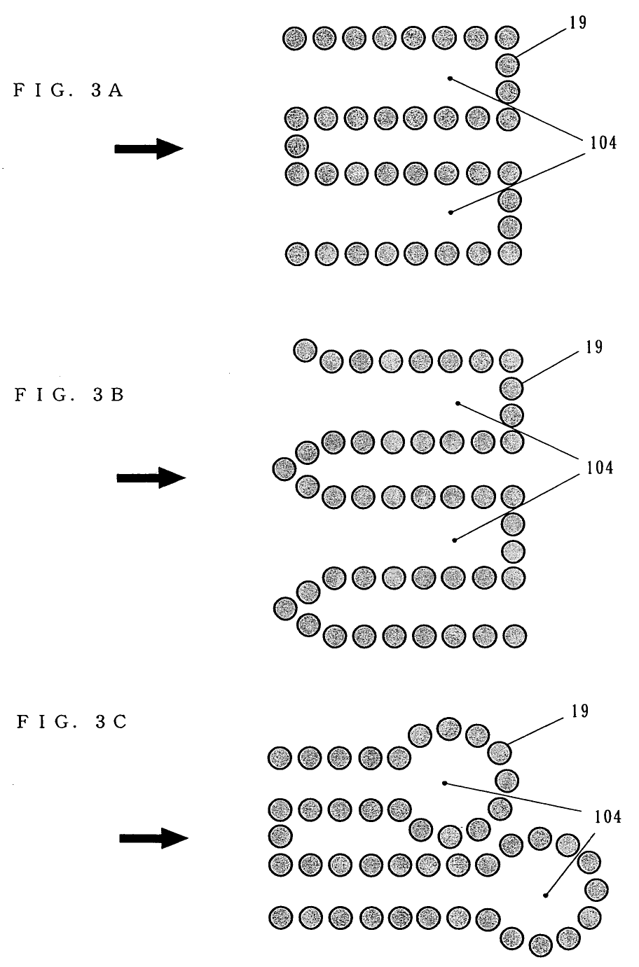

The cavity 104 is typically in the shape of substantially a rectangular parallelepiped as shown in FIGS. 1 or 2. The present invention is not limited to this. FIG. 3 is a schematic diagram showing the column-shaped structures 19 and variations of the arrangement thereof. FIGS. 3A to 3C each show the cavity 104 viewed from the top. An arrow in FIG. 3 indicates a direction in which a sample liquid flows.

FIG. 3A shows a cavity having substantially the same rectangular parallelepiped shape as that of FIGS. 1 and 2, except that each column-shaped structure has a circular cross-section (i.e., each structure is in the shape of a cylinder).

FIGS. 3B and 3C show that cylinder-shaped structures 19 similar to those of FIG. 3A are arranged in a folded line, but are different from FIG. 3A in the shape of the arrangement. When the mouth of the cavity is wide open as shown in FIG. 3B, it is easier for blood cells to enter the cavity and blood cells are prevented from being held back in the vicinity of the mouth. In FIG. 3C, the shape of a cavity mouth is similar to those of FIGS. 1, 2 and 3A, but FIG. 3C is different from FIGS. 1, 2 and 3A in that a bottom portion of the cavity is in the shape of a circle.

As described above, there are various possible cavity shapes. The present invention is not limited to those illustrated herein.

The efficiency of filtration can be further improved by increasing the number of folds in the array of the structures 19 within the limited space of the channel 18. Typically, assuming that the channel has a width of 1.5 mm, 750 cavities each having a width 10 μm, which is in the shape of substantially a rectangular parallelepiped, can be arranged at intervals of 10 μm. The number of cavities may be greater or smaller than 750.

The widths α, β and γ may be changed, depending on the size of a blood cell of a subject. For example, the size of a red blood cell usually varies between male and female (a female red blood cell is smaller). Therefore, when a female blood sample is used, a value smaller than the above-described typical value can be used.

The amount of a sample required for filtration using the filter of the present invention may vary depending on the channel depth, the structure arrangement or the like. Therefore, the optimal amount of a sample may be determined as appropriate by one of ordinary skill in the art, depending on the size of a filter or the like. In the filter of the present invention, there is no absorption by a filter material which is observed in conventional technologies. Also in the filter of the present invention, filtration is not performed by the effect of delayed movement of blood cells. Therefore, the filter size can be reduced. Therefore, the amount of a sample can be reduced as compared to that of conventional technologies.

The filtration efficiency of the filter of the present invention depends on the arrangement of the column-shaped structures, the shape of the cavity, the inner volume of the cavity or the like. Therefore, these parameters need to be optimized as appropriate.

A cross-section of the column-shaped structure may be in any shape, such as, for example, a quadrangle, a circle or the like. However, in order to prevent the membrane of a red blood cell from being destroyed (lysis) to the extent possible, it is preferable that the column-shaped structure has a smooth surface (e.g., a circular cross-section).

The column-shaped structure and the channel inner wall are preferably made of a material which suppresses blood coagulation, such as silicone resin, Teflon, epoxy resin or the like, or are preferably covered with such a material. Alternatively, the column-shaped structure and the channel inner wall may be made of a high-purity glass layer, such as SiO₂ or the like, which contains a small amount of Ca, which promotes blood coagulation.

The inner height (or thickness) of the channel 18 or the cavity 104 may be any value, and taking the current manufacturing technology into consideration, is practically smaller than or equal to about 100 μm. However, the thickness (height) may be greater than about 100 μm, depending on the state of the art.

The filter of the present invention is not limited to those which are produced on a substrate using a semiconductor processing technique. For example, the filter of the present invention may be similarly produced using a plastic molding technique.

The present invention also provides a biosensor comprising the above-described filter.

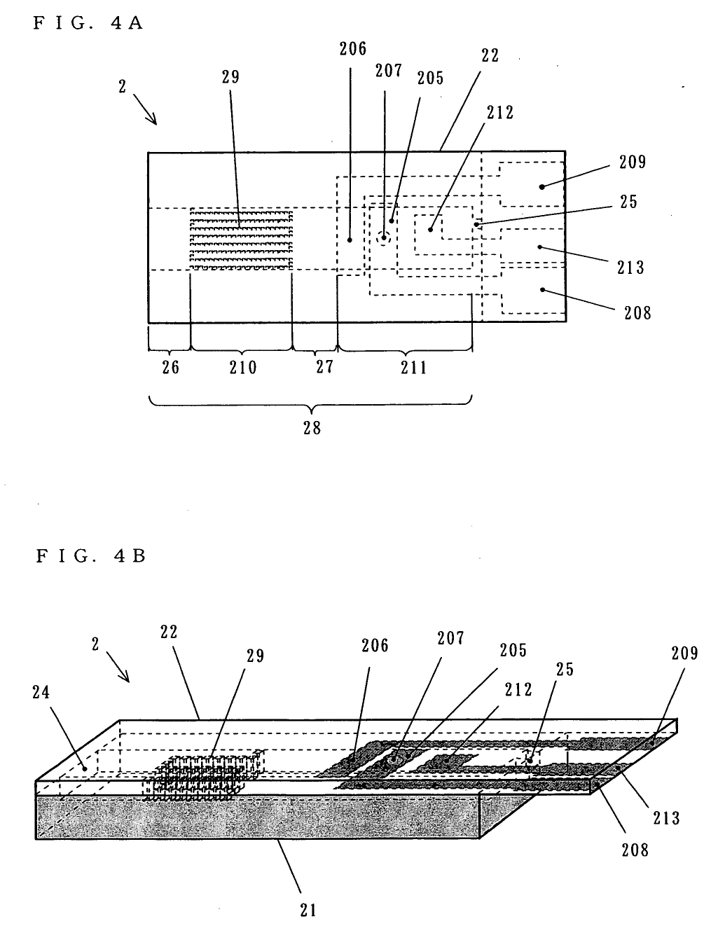

FIG. 4 shows a structure of a biosensor 2 according to the present invention in which a filter for separating blood cells from collected blood is integrated with a biosensor for analyzing a test item, such as a blood sugar value or the like. FIG. 4A is a schematic top view of the biosensor 2. FIG. 4B is a perspective view thereof.

Referring to FIGS. 4A and 4B, the biosensor 2 with built-in filter of the present invention comprises a substrate 21, a cover 22 attached to the substrate 21, and a channel 28 defined by the substrate 21 and the cover 22. The biosensor 2 further comprises a sample inlet 24 for introducing a sample, which is formed at one end of the channel 28, and an air escape opening 25 for causing the air to escape from a channel, which is formed at the other end.

The biosensor 2 further comprises: a filter region 210 including a plurality of column-shaped structures 29; a reservoir 26 for retaining a blood sample containing the blood cell component, which is located upstream of the filter region 210; a reaction region 211 accommodating a working electrode 205, a counter electrode 206, and a liquid introduction detecting electrode 212 for detecting the arrival of a sample to the reaction region 211, the reaction region 211 being located downstream of the filter region 210; and a sample introduction pathway 27 connecting between the filter region 210 and the reaction region 211. The filter region 210, the reservoir 26, the reaction region 211 and the sample introduction pathway 27 are formed on the channel 28. On the working electrode 205, a reagent 207 containing an enzyme (e.g., glucose oxidase) and a mediator (e.g., a metal complex, such as ferricyanate ion) is provided. The channel 28 and the column-shaped structure 29 may be formed on the substrate 21 using a semiconductor processing technique, such as reactive ion etching.

The biosensor 2 further comprises a lead 208 which is integrally connected to the working electrode 205, a lead 209 which is integrally connected to the counter electrode 206, and a lead 213 which is integrally connected to the liquid introduction detecting electrode 212. The electrodes (205, 206, 212) and the leads (208, 209, 213) may be formed on a surface of the cover 22 facing the substrate 21 using a technique, such as sputtering deposition or the like.

The thus-constructed biosensor 2 with built-in filter of the present invention can have a size similar to that of conventional biosensors without a filter, and can analyze a blood sugar value or the like quickly using a trace amount of sample.

Hereinafter, the present invention will be described in more detail by way of examples. The scope of the present invention is not limited to the examples.

### EXAMPLE 1

Separation of Blood Components Using a Filter Produced Herein

A filter based on the present invention was produced on a silicon substrate. Separation of blood components was observed. Hereinafter, this process will be described with reference to FIGS. 1 and 2.

A silicon substrate 11 having a size of 5 mm×9 mm×0.5 mm was subjected to reactive ion etching. Thereby, a channel 18 and a plurality of column-shaped structures 19 were formed on substantially a middle of the substrate 11, providing a plurality of folded portions. Further, an oxide film (not shown) was formed on surfaces of the column-shaped structure 19 and the silicon substrate 11 by thermal oxidation. A silicone resin cover 12 is tightly attached onto the substrate 11. As a result, a filter 1 of the present invention was produced.

In the filter 1 thus produced, the channel 18 had a width of 1.5 mm, a height of 30 μm, and a length of 9 mm. Cavities 104 formed by a number of folds of the column-shaped structures 19 and an inner wall of the channel 18 are each in the shape of substantially a rectangular parallelepiped. A depth of each cavity was 2 mm, and a width β of a cavity mouth was 10 μm. The column-shaped structures 19 each had a square cross-section of 2 μm×2 μm and were arranged at a pitch of 4 μm to form slits 101 and 103 having a width of 2 μm. Also, 750 folds were arranged at a pitch of 20 μm in a direction perpendicular to a longitudinal direction of the channel.

Note that the oxide film may be formed using reduced pressure CVD (chemical vapor deposition), plasma CVD, atmospheric CVD or the like instead of thermal oxidation.

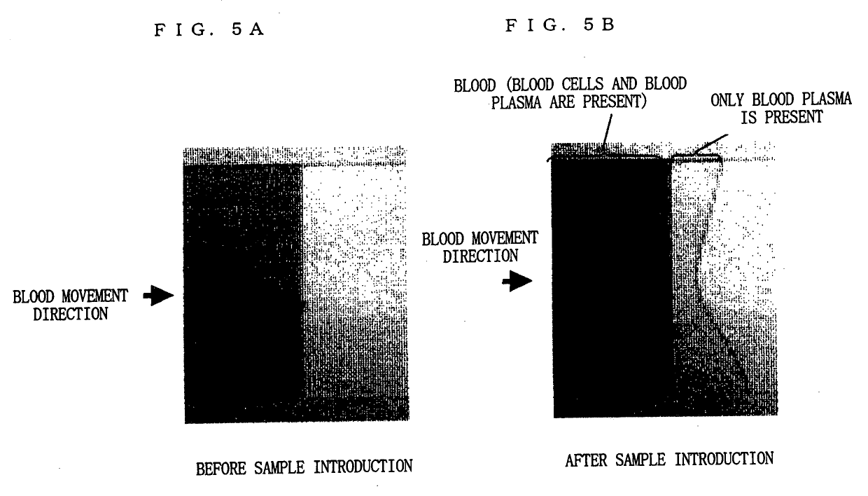

The thus-produced blood component separation filter 1 of the present invention was evaluated by the following procedure. Initially, the filter 1 thus produced was placed on a stage of a microscope. A blood sample was dropped onto a sample inlet 14 which is provided at one end of the channel of the filter 1. The flow of the liquid was recorded by a microscope video camera. FIG. 5 shows a movement of the liquid.

FIG. 5A shows a state of the filter 1 before introduction of a sample. FIG. 5B shows a state of the filter 1 after introduction of the sample. In the photographs of FIG. 5, arrows indicate a direction in which the blood travels. In each photograph, a dark portion on the left-hand side indicates a filter region 110, while a light portion on the right-hand side indicates a reservoir 17 for a sample which has passed through the filter region 110. The blood travels from the left to the right of the photograph by the capillary action.

The blood was introduced from the sample inlet 14, and at the same time, the blood was loaded in a reservoir 16 closer to the sample inlet 14 of the filter 1. Thereafter, the blood was loaded in the filter region 110.

As shown in FIG. 5B, after introduction of the sample, transparent blood plasma thus filtered flowed from the filter region 110 into a downstream reservoir 17. A border line seen in the light portion of the photograph is a front line of the sample liquid containing only blood plasma after separation of blood cells from the blood. An average time required from blood introduction to completion of blood plasma filtration was only 25 seconds. Thus, blood cells and blood plasma in the blood could be separated efficiently.

### EXAMPLE 2

Production of the Biosensor 2 of the Present Invention

Example 2 will be described with reference to FIGS. 4A and 4B. As a substrate 21, a silicon substrate (P type, (100) surface, diameter: 100 mm, thickness: 525 μm, resistivity: 10 to 20 Ω·cm, manufactured by Shin-Etsu Silicon) was used. On the substrate 21, a channel 28 and a plurality of column-shaped structures 29 were formed using a semiconductor processing technique, such as reactive ion etching or the like, providing a number of folds in an arrangement of the column-shaped structures 29. Next, an insulating film was formed on a surface of the silicon substrate 21 by thermal oxidation.

On the other hand, a working electrode 205, a counter electrode 206, a liquid introduction detecting electrode 212, and leads 208, 209 and 213 were produced on one side of a cover 22 of a resin sheet by sputtering deposition. Thereafter, glucose oxidase and a mediator (e.g., a metal complex, such as ferricyanate ion) were placed as a reagent 207 on the working electrode 205 by a dispense method using a syringe. The cover 22 was attached to the substrate 21 by thermal pressure bonding, where the side of the cover 22 on which the electrodes (205, 206, 212) were formed faced the substrate 21. As a result, a sample channel 28, a sample inlet 24 for introduction of a sample (at one end of the channel), and an air escape opening 25 for causing the air to escape (at the other end of the channel) are defined.

A substance to be measured contained in blood plasma is measured in a region (reaction region) 211 in which the electrodes (205, 206, 212) and the reagent 207 are provided. Specifically, the reagent 207 provided on the working electrode 205 is dissolved as the blood is introduced, and the enzyme in the reagent 207 reacts with glucose in the blood. A voltage of 0.5 V is applied between the working electrode 205 and the counter electrode 206. A current flowing between these electrodes is measured. A glucose value is calculated based on a current value after a predetermined time (e.g., 30 seconds) has passed.

The biosensor 2 obtained by the above-described method has a length of 11 mm, a width of 5 mm, and a height of 2.5 mm. The channel 28 has a width of 1.5 mm, a length of 7.0 mm, and a height of 30 μm. A required amount of a sample is smaller than 31.5 nl. Thus, according to the present invention provides a biosensor 2 with built-in filter which requires a sample amount smaller than that of conventional blood component separation filters.

### EXAMPLE 3

Measurement of Glucose Concentration

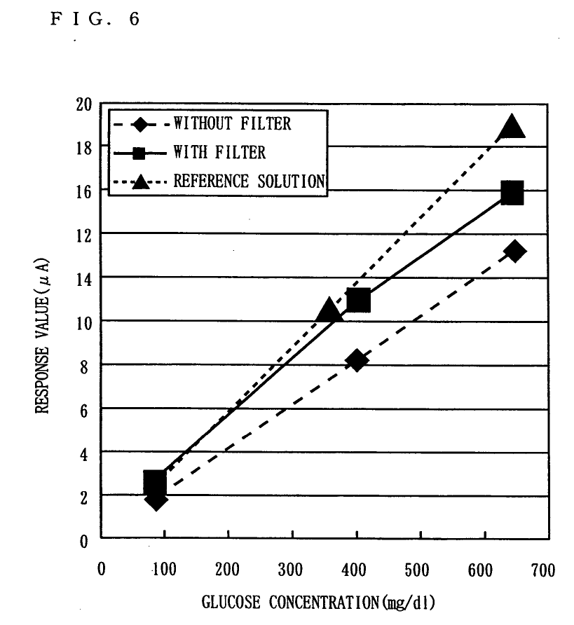

FIG. 6 shows comparison of sensitivity of biosensors over a blood glucose concentration range of 87 to 648 mg/dl. In FIG. 6, a closed triangle indicates a reference solution, a closed square indicates measurement data of the biosensor 2 with built-in filter of the present invention, and a closed diamond indicates measurement data of a biosensor without a filter as a comparative example.

Blood samples having different glucose concentrations were prepared by adding glucose solution to a blood sample (hematocrit value: 44) to a concentration of 87 to 648 mg/dl. The reference solution was prepared by dissolving glucose in phosphate buffer saline.

The biosensor 2 was produced as shown in Example 2. The biosensor without a filter (comparative example) was of a type which requires 600 nl of sample.

The blood was introduced through the sample inlet 24, and after 25 seconds, a voltage of 0.5 V was applied between the lead 208 of the working electrode 205 and the lead 209 of the counter electrode 206. After 5 seconds, a current value was measured.

The response value of the device 2 with built-in filter of the present invention was increased by about 20% as compared to that of the device without a filter, and was closer to the response value of the glucose reference solution. Therefore, it will be understood that the device 2 with built-in filter of the present invention achieved improved sensitivity.

### EXAMPLE 4

Measurement of Cholesterol Concentration

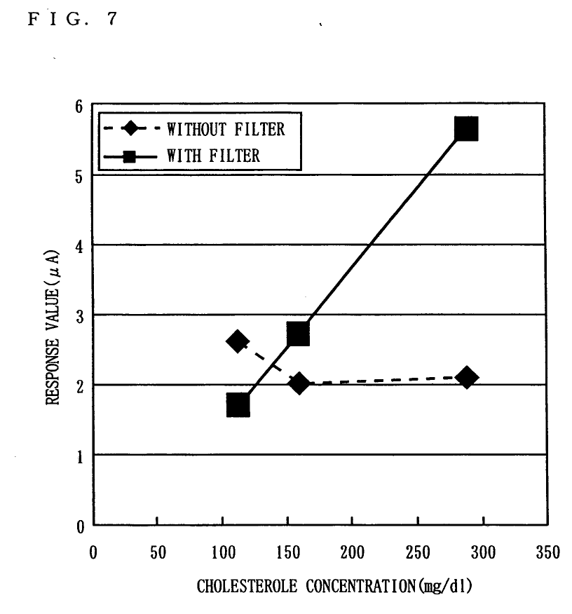

FIG. 7 shows comparison of sensitivity of biosensors over a blood cholesterol concentration range of 113 to 288 mg/dl. In FIG. 7, a closed square indicates the biosensor 2 with built-in filter of the present invention, and a closed diamond indicates a biosensor without a filter as a comparative example.

Blood samples having different cholesterol were prepared by removing blood plasma from the whole blood by centrifugation, and thereafter, adding a reference serum (Seraclear-LP abnormal band, manufacture by Azwell) having a high cholesterol value to the blood sample. Note that cholesterol esterase was used as a reagent in Example 4.

As shown in FIG. 7, blood cells functioned as an interfering material in the biosensor without a filter, so that a current proportional to a cholesterol concentration was not obtained, i.e., a cholesterol concentration could not be measured. In the case of the biosensor 2 with built-in filter, a value dependent on a cholesterol concentration was measured. Therefore, it will be understood that accurate measurement could be achieved only after the device 2 with built-in filter is used.

Although Examples 3 and 4 illustrate measurement using the enzyme electrode method, other measuring means may be used instead of the enzyme electrode method. An example of such a measuring means is an enzyme color test method.

### INDUSTRIAL APPLICABILITY

As described above, a filter of separating blood cells and blood plasma according to the present invention is useful as a biosensor or a pretreatment device for a clinical testing, such as DNA diagnosis or the like.

1. A filter for filtering a blood sample containing a blood cell component, comprising:
a channel for causing the blood sample to flow;
an opening for introducing the blood sample, the opening being located at one end of the channel; and
an opening for discharging the blood sample filtered through the channel, the opening being located at the other end of the channel,
wherein a plurality of structures having a cylinder shape are disposed in the channel to prevent the blood cell component from passing through the channel,
the structures are disposed at intervals such that a slit through which the blood cell component cannot pass is formed between each structure and an adjacent inner wall of the channel or between adjacent structures, and
the plurality of structures and the inner wall of the channel define at least one cavity functioning as a blood cell reservoir for accommodating the blood cell component in the channel.

2. The filter according to claim 1, wherein at least two cavities are defined in the channel.

3. The filter according to claim 1, wherein a depth of the cavity is greater than a width of a mouth of the cavity.

4. The filter according to claim 1, wherein a width of a mouth of the cavity is in a range of about 2 μm to about 10 μm.

5. The filter according to claim 1, wherein the cavity is in a shape of substantially a rectangular parallelepiped.

6. The filter according to claim 1, wherein a width of the slit is in a range of about 0.1 μm to about 2 μm.

7. The filter according to claim 1, wherein the channel is formed by a substrate, a spacer, and a cover attached to the substrate via the spacer.

8-9. (canceled)

10. The filter according to claim 1, wherein the blood sample is introduced into the channel by capillary action.

11. The filter according to claim 1, wherein the structure and the inner wall of the channel are made of silicone resin, Teflon or epoxy resin, or surfaces of the structure and the inner wall of the channel are covered with any of silicone resin, Teflon and epoxy resin.

12. A biosensor having a filter region for filtering a blood sample containing a blood cell component, comprising:
a substrate;
a measuring system supported by the substrate;
a reagent system containing a redox enzyme supported by the measuring system or the substrate in the vicinity of the measuring system;
a cover combined with the substrate to define, between the cover and the substrate, a filter region for removing the blood cell component from the blood sample, a reaction region for accommodating the measuring system and the reagent system, and a sample introduction pathway connected to the filter region for introducing the sample to the reaction region,
the filter region is defined by:
a channel for causing the blood sample to flow;
an opening for introducing the blood sample, the opening being located at one end of the channel;
an opening for discharging the filtered blood sample, the opening being located at the other end of the channel and being connected to the sample introduction pathway; and
a plurality of structures disposed in the channel to prevent the blood cell component from passing through the channel,
the structures are disposed at intervals such that a slit through which the blood cell component cannot pass is formed between each structure and an adjacent inner wall of the channel or between adjacent structures, and
the plurality of structures and the inner wall of the channel define at least one cavity functioning as a blood cell reservoir for accommodating the blood cell component in the channel.

13. The biosensor according to claim 12, wherein the measuring system includes an electrode system comprising at least a pair of electrodes.

14. The biosensor according to claim 12, wherein at least two cavities are defined in the channel.

15. The biosensor according to claim 12, wherein a depth of the cavity is greater than a width of a mouth of the cavity.

16. The biosensor according to claim 12, wherein a width of a mouth of the cavity is in a range of about 2 μm to about 10 μm.

17. The biosensor according to claim 12, wherein the cavity is in a shape of substantially a rectangular parallelepiped.

18. The biosensor according to claim 12, wherein a width of the slit is in a range of about 0.1 μm to about 2 μm.

19. The biosensor according to claim 12, wherein the channel is formed by a substrate, a spacer, and a cover attached to the substrate via the spacer.

20. The biosensor according to claim 12, wherein the structure is in a shape of a column.

21. The biosensor according to claim 20, wherein the structure is in a shape of a cylinder.

22. The biosensor according to claim 12, wherein the blood sample is introduced into the channel by capillary action.

23. The biosensor according to claim 12, wherein the structure and the inner wall of the channel are made of silicone resin, Teflon or epoxy resin, or surfaces of the structure and the inner wall of the channel are covered with any of silicone resin, Teflon and epoxy resin.

24. The filter according to claim 1, wherein a cross-sectional shape parallel to a depth direction and a width direction of the cavity is substantially a circle at a bottom portion of the cavity.

25. The filter according to claim 1, wherein the plurality of structures has a folded structure which forms the cavity and are disposed across an entire width of the channel.

26. The filter according to claim 25, wherein the slit is also formed between the structure and an inner wall of the channel, the inner wall defining the width of the channel.
